(19) United States
(12) Patent Application Publication
(10) Pub. No.: US 2022/0399099 A1
(43) Pub. Date: Dec. 15, 2022

(54) SYSTEM AND METHOD FOR PROVIDING PERSONALIZED DIETARY SUGGESTION PLATFORM

(71) Applicant: SEOUL NATIONAL UNIVERSITY R&DB FOUNDATION, Seoul (KR)

(72) Inventor: Jiyoung KIM, Seoul (KR)

(21) Appl. No.: 17/838,055

(22) Filed: Jun. 10, 2022

(30) Foreign Application Priority Data
Jun. 15, 2021 (KR) 10-2021-0077354
Apr. 4, 2022 (KR) 10-2022-0041661

(51) Int. Cl. G16H 20/60; G16H 50/20; G16H 10/60; G16H 40/67

(52) U.S. Cl. CPC G16H 20/60; G16H 50/20; G16H 10/60; G16H 40/67

(57) ABSTRACT

Provided is a method and a system for providing personalized dietary suggestion platform and provides a method and a system for providing personalized dietary suggestion platform in which a complex system is constructed by using user's health-related data, food product data, human physiology data, and environmental data, and a user-personalized diet is derived by learning thereof.

### BACKGROUND

### 1. Technical Field

An embodiment of the present disclosure relates to a system and a method for providing a personalized dietary suggestion platform providing system and relates to a technology for suggesting a customized nutritional ingredients or food required for a user and relaying the sale of the suggested food.

### 2. Related Art

Advances in genomics and omics technology enable a collection of personal health big data, and a new era called “personalized” or “precision medicine” considering individual diversity in disease treatment and prevention of disease is ushered in.

A paradigm shift from public health care to personalized health care is starting. The United States is conducting a government-led medical information data standardization project in 2018. The National Institute of Health is constructing a large cohort of health-related data from more than 1 million different individuals through All of Us program. In addition, personal data with various health conditions is collected, and bioresource data is constructed as a public research resource at the national level and provided to researchers as research information.

Science, which is a world-renowned scientific journal, introduced a study on precision nutrition (personalized diet) targeting 10,000 people worth 150 billion Korean won for five years under research at the National Institute of Health in the United States in February 2021. According to the results of a recent study, predicting a health change is higher in level when several environmental factors are considered together than predicting a health change when only genetic factors are considered. This precise approach to disease leads to new insights into disease outbreak research, and it is necessary to develop a technology for how to suggest a personalized disease-preventing diet such as precise health care, precise nutrition, and precise food.

Interest in health is growing the market for ‘personalized health care’ or ‘personalized food’ in line with the personal-oriented consumption trend. Accordingly, data related to personal health are separately collected and managed.

Personal health data are scattered and managed, such as individual hospital treatment records, test results, consultation records, pulse, blood sugar, health checkup data, food purchase data, meal and water intake, nutritional supplements and medication record data, sleep data, exercise and activity data, family disease history (gene/lifestyle) data, genetic data, defecation data, and microbiome data. Therefore, user convenience and health-related analysis accuracy are reduced. Accordingly, it is necessary to develop a technology capable of integrating and analyzing scattered data related to personal health to increase user convenience and improve health-related analysis and prediction accuracy.

### SUMMARY

A system and a method for providing a personalized dietary suggestion platform according to an embodiment of the present disclosure are for recommending personalized nutritional ingredients, food, and diet that may reduce the risk of disease by considering a user's health, genetics, and environmental factors in a complex manner.

However, a technical task to be achieved by the present embodiment is not limited to the technical task as described above, and other technical tasks may exist.

As technical means for achieving the technical task described above, a method for providing a personalized dietary suggestion platform performed by a processor according to an embodiment includes a step of acquiring health-related data related to the user's health; a step of constructing a database by using big data including food product data, human physiology data, and environmental data, and the health-related data; and a step of suggesting a personalized nutritional ingredient including any one or more of a health maintenance ingredient, a disease nutritional ingredient, and a physiologically active ingredient tailored to user's health-related characteristics, or a food product including the personalized nutritional ingredient by executing a personalized dietary derivation model learned by using the database, in which the food product data includes data related to a nutritional ingredient of a food product and characteristics of the food product, the human physiology data includes data related to body, disease, and genetic information, and the environmental data includes data related to a climate environment and an environmentally hazardous substance.

In addition, a system for providing a personalized dietary suggestion platform according to an embodiment includes a memory in which a personalized dietary suggestion program is stored; and a processor that executes the personalized dietary suggestion program stored in the memory, in which the processor executes the personalized dietary suggestion program, acquires health-related data related to user's health, constructs a database using big data including food product data, human physiology data, and environmental data, and health-related data, and suggests a personalized nutritional ingredient including any one or more of a health maintenance ingredient, a disease nutritional ingredient, and a physiologically active ingredient tailored to a health-related characteristic of the user or a food product including the personalized nutritional ingredient by executing a personalized dietary derivation model learned by using the database, and in which the food product data includes data related to a nutritional ingredient and a characteristic of the food product, the human physiology data includes data related to a body, a disease, and genetic information, and the environmental data includes data related to a climatic environment and an environmentally hazardous substance.

The system and the method for providing personalized dietary suggestion platform according to the embodiments of the present disclosure may recommend a personalized menu capable of reducing the risk of disease by considering a user's health, genetics, and environmental factors in a complex manner.

### BRIEF DESCRIPTION OF THE DRAWINGS

Various embodiments of the present disclosure will become more apparent in view of the attached drawings and accompanying detailed description, in which:

FIG. 1 is a structural diagram of a system for providing a personalized dietary suggestion platform according to an embodiment of the present disclosure;

FIG. 2 is a configuration diagram of a server of the system for providing a personalized dietary suggestion platform according to an embodiment of the present disclosure;

FIG. 3 is a conceptual diagram of a server according to an embodiment of the present disclosure; and

FIG. 4 is a flowchart of a method for providing a personalized dietary suggestion platform according to an embodiment of the present disclosure.

### DETAILED DESCRIPTION OF THE EMBODIMENTS

Hereinafter, the present disclosure will be described in detail with reference to the accompanying drawings. However, the present disclosure may be implemented in various different forms and is not limited to the embodiments described herein. In addition, the accompanying drawings are only for easy understanding of the embodiments disclosed in the present specification, and the technical ideas disclosed in the present specification are not limited by the accompanying drawings. All terms including technical terms and scientific terms used herein should be interpreted as meanings commonly understood by those of ordinary skill in the art to which the present disclosure belongs. Terms defined in the dictionary should be interpreted as having additional meanings consistent with the related technical literature and the presently disclosed content and are not interpreted in a very ideal or limiting sense unless otherwise defined.

In order to clearly explain the present disclosure in the drawings, parts irrelevant to the description are omitted, and the size, form, and shape of each component shown in the drawings may be variously modified. The same/similar reference numerals are attached to the same/similar parts throughout the specification.

The suffixes “module” and “portion” for components used in the following description are given or mixed in consideration of only the ease of writing the specification, and do not have distinct meanings or roles by themselves. In addition, in describing the embodiments disclosed in the present specification, when it is determined that detailed descriptions of related known technologies may obscure the gist of the embodiments disclosed in this specification, detailed descriptions thereof are omitted.

Throughout the specification, when a part is said to be “connected (joined, contacted, or coupled)” with another part, this means not only when it is “directly connected (joined, contacted, or coupled)”, but also refers to another member in the middle. It includes a case of “indirectly connected (joined, contacted, or combined)” between them. Also, when a part “includes (provides or has)” a component, it does not exclude other components unless otherwise stated, but further “includes (provides or has)” other components.

Terms indicating an ordinal number such as first, second, etc. used herein are used only for the purpose of distinguishing one component from another, and do not limit the order or relationship of the components. For example, a first component of the present disclosure may be referred to as a second component, and similarly, a second component may also be referred to as a first component. Forms of the singular expression used herein should be construed to include forms of the plural expression as well, unless the meaning is clearly opposite.

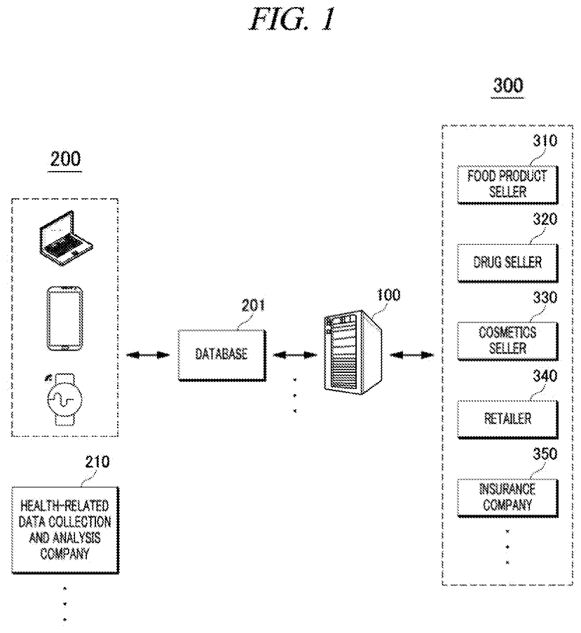

Hereinafter, a system for providing a personalized dietary suggestion platform (hereinafter, referred to as a “personalized dietary suggestion platform providing system”) according to an embodiment of the present disclosure will be described with reference to FIG. 1.

As shown in FIG. 1, a personalized dietary suggestion platform providing system 100 may perform data transmission and reception with a user terminal 200, a health-related data collection company 210 or an external database 201 via a communication network. Accordingly, the personalized dietary suggestion platform providing system 100 may receive data for the personalized dietary suggestion of the user from the user terminal 200, the health-related data collection company 210, or the external database 201. In addition, the personalized dietary suggestion platform providing system 100 may transmit a personalized dietary suggestion result generated by using the received data to the user by using the user terminal 200.

For example, the user terminal 200 may mean all types of handheld-based wireless communication devices such as a notebook on which a web browser is mounted, a desktop (desktop), a laptop (laptop), wireless communication device in which portability and mobility are guaranteed, a smart phone, and a tablet PC. In addition, the user terminal 200 may mean a device capable of acquiring the user's body information or activity information, or displaying the result of the personalized dietary suggestion, such as a smart device, a smart watch, and a wearable device.

The health-related data collection and analysis company 210 may mean a company that is related to blood indicator tests such as biomicrobiome analysis, continuous blood glucose analysis, and blood lipid analysis, and a company that collects and analyzes data related to a user's health such as hair mineral test, wearable device data analysis, smart device, heart rate data analysis, sleep data analysis, and dietary survey analysis.

The personalized dietary suggestion platform providing system 100 may acquire data related to the user's health from any one or more of the user terminal 200, the user's health-related data collection company 210, and the external database 201.

The external database 201 may mean a cloud, a server, or a database in which data required to generate the personalized dietary suggestion is stored. The external database 201 may mean a user's smart device, a wearable device, a cloud, a server, or a database in which information related to and user's health is stored. In addition, the external database 201 may mean a cloud, a server, or a database in which data directly or indirectly related to the user's health, such as food product data, human physiology data, and environmental data, is stored.

For example, the external database 201 may mean a cloud, a server, or a database of a company related to blood indicator tests such as vicrobiome analysis, continuous blood glucose analysis, and blood lipid analysis, and the user's health-related data collection and analysis company 200 performing analyses such as hair mineral test, wearable device data analysis, smart device, heart rate data analysis, sleep data analysis, and dietary survey analysis.

In addition, the external database 201 may mean a cloud, a server, or database where health-related data is stored which is collected from public institutions, medical institution medical records, lifelogs, health management companies, and the like, acquired by My Health Way (my data in the medical field) constructed by the 4th Industrial Revolution Committee and related ministries (Ministry of Health and Welfare, Ministry of Science and Technology Information and Communication, Ministry of Trade, Industry and Energy Ministry of Trade, and Personal Information Protection Committee) in Korea.

Here, the food product data may include data related to a nutritional ingredient of a food product and characteristics of the food product. The human physiology data may include data related to body, disease, and genetic information, and the environmental data may include data related to the climate environment and environmentally hazardous substances.

In addition, the personalized dietary suggestion platform providing system 100 may provide the user with a list of recommended purchases for food, medicine, cosmetics, healthcare products, healthcare services, insurance, and the like, or may perform a function of relaying between a seller (distributor and insurance companies) and the user, by using a result of the personalized dietary suggestion which is derived.

For example, the personalized dietary suggestion platform providing system 100 may display preferentially, to the user, a seller 300 based on preset criteria or according to the weight to suggest a purchase of food, medicine, cosmetics, health care products, health care services, insurance, and the like by using the result of the personalized dietary suggestion. In addition, the personalized dietary suggestion platform providing system 100 may provide the user with the product of the seller 300 registered in the personalized dietary suggestion platform providing system 100.

Here, the seller 300 may include not only a food product seller 310, but also a drug seller 320, a cosmetics seller 330, a retailer 340, a healthcare company 350, an insurance company 360, and the like. In other words, products related to the user's diet, such as agricultural and livestock products, processed food, cooked food, meal kits, diet provision, cooking method provision, hospitals, catering companies, restaurant companies, health functional foods, nutritional supplements, smart farms, cosmetics, drugs, and herbal medicines, all products providing services, and service providers may be a target.

The personalized dietary suggestion platform providing system 100 may transmit a personalized dietary list to a retailer mainly used by the user or a convenience store located in the customer's movement. Accordingly, the retailers and convenience stores may prepare products included in the user's personalized dietary list and deliver them to the user, or the user may pick up the products.

In addition, the user of the personalized dietary suggestion platform providing system 100 may include autistic infants and toddlers, patients, doctors, and hospitals. In this case, the personalized dietary suggestion platform providing system 100 may be used as a decision support system, a software medical device, and a digital treatment device that provides a diet for disease prevention, management, and treatment of autistic children and patients.

The personalized dietary suggestion platform providing system 100 can provide personalized diets to prevent diseases such as Eating Disorders, Cognitive Disorders, Dementia, Alzheimer's Disease, Parkinson's Disease, AHDH, Schizophrenia, Depression, High Blood Pressure, Hyperlipidemia, Cardiovascular Disease, Obesity, Diabetes, Weight Loss, Metabolic Disease, Kidney Disease and Cancer.

For example, if the autistic infants and toddlers are the users, the personalized dietary suggestion platform providing system 100 may acquire health-related data for the autistic infants and toddlers based on the analysis of dietary taste, habit, intake nutritional information, intake method, intake time, food photos before and after food intake, intake video, blood, urine, feces, hair, and the like. In addition, the personalized dietary suggestion platform providing system 100 may derive a list of care foods for the autistic infants and toddlers based on neurobehavioral developmental abnormality symptoms obtained from nutritional ingredients, metabolites, intestinal microbes, genes, heavy metal information, intake videos, dietary preferences, habits, and the like identified in a sample.

The user terminal 200, the health-related data collection company 210, and the external database 201 may perform data transmission/reception with the personalized dietary suggestion platform providing system 100 via a communication network. The communication network refers to a connection structure capable of exchanging information between nodes, such as a network or a plurality of terminals and servers. Accordingly, the communication network includes a local area network (LAN), a wide area network (WAN), the Internet (WWW), a wired/wireless data communication network, a telephone network, a wired/wireless television communication network, and the like. An example of the wireless data communication network includes 3G, 4G, 5G, 3rd Generation Partnership Project (3GPP), Long Term Evolution (LTE), World Interoperability for Microwave Access (WIMAX), Wi-Fi, Bluetooth communication, infrared communication, ultrasound communication, Visible Light Communication (VLC), LiFi, or the like, but is not limited thereto.

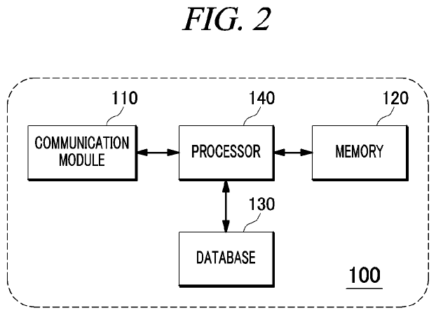

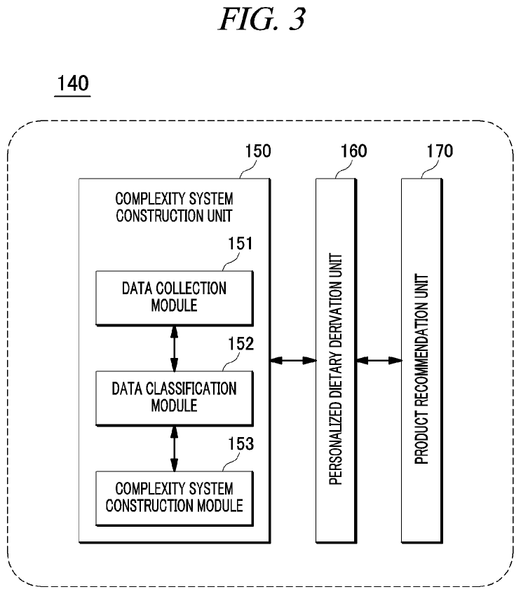

Hereinafter, a configuration of the personalized dietary suggestion platform providing system 100 according to the embodiment of the present disclosure will be described with reference to FIGS. 2 and 3.

The personalized dietary suggestion platform providing system 100 is configured to include a communication module 110, a memory 120, and a processor 140, and may further include a database 130. The data stored or generated in the user terminal 200, the health-related data collection company 210, and the external database 201 is transmitted to the communication module 110 included in the personalized dietary suggestion platform providing system 100 by using a communication network.

The communication module 110 included in the personalized dietary suggestion platform providing system 100 provides a communication interface which is required to provide a signal, in a form of data, which is transmitted and received signals to and from the user terminal 200, the health-related data collection company 210, and the external database 201 in association with the communication network. Here, the communication module 110 may be a device including hardware and software necessary for transmitting and receiving signals such as control signals or data signals through wired/wireless connection with other network devices.

A personalized dietary suggestion program for providing the personalized diet may be stored in the memory 120. The personalized dietary suggestion program stored in the memory 120 may be driven by the processor 140.

In addition, the memory 120 performs a function of temporarily or permanently storing data processed by the processor 140. Here, the memory 120 may include a volatile storage medium or a non-volatile storage medium, but the scope of the present disclosure is not limited thereto. In addition, the database 130 may constitute a part of the memory 120, but not necessarily located inside the personalized dietary suggestion platform providing system 100 but may be located outside.

The memory 120 may store a separate program such as an operating system for processing and controlling the processor 140 or may perform a function of temporarily storing input or output data. In addition, the database 130 may perform a function of long-term storage of data input or output to the processor 140.

The memory 120 may include at least one type of storage medium of a flash memory type, hard disk type, multimedia card micro type, card type memory (for example, SD or XD memory, or the like), RAM, and ROM. In addition, the personalized dietary suggestion platform providing system 100 may operate a web storage that performs a storage function of the memory 120 on the Internet.

The processor 140 executes the program stored in the memory 120 and performs the process corresponding to each operation of the personalized dietary suggestion program which will be described below.

The processor 140 executes the personalized dietary suggestion program stored in the memory 120, and controls overall operations for providing the personalized diet. The personalized dietary suggestion service is a service that means a service that provides a purchase association interface to suggest nutritional ingredients or food to maintain or improve user's health, or to purchase personalized food by using a complex system configured of various factors that affect health, such as health-related data, food product data, human physiology data, and environmental data which are related to the health of the user collected from various institutions or devices.

To this end, the processor 140 may be implemented including at least one processing unit (CPU, micro-processor, DSP, or the like), a random access memory (RAM), a read-only memory (ROM), and the like, and may perform the program stored in the memory 120 through at least one processing unit by being read by the RAM. In addition, according to an embodiment, the term ‘processor’ may be interpreted as the same meaning as terms such as ‘controller’, ‘arithmetic unit’, and ‘control unit’.

Here, the processor 140 may perform various functions according to the execution of the program stored in the memory 120, and the detailed modules included in the processor 140 may be represented by a complexity system construction unit 150 and a personalized dietary derivation unit 160 according to each function. A detailed module included in the processor 140 will be described in detail with reference to FIG. 3 which is described later.

Referring to FIG. 3, the personalized dietary suggestion platform providing system 100 may include the complex system construction unit 150, the personalized dietary derivation unit 160, and a product recommendation unit 170.

The complex system construction unit 150 uses health-related data, food product data, human physiology data, and environmental data collected from the user terminal 200, the health-related data collection company 210, and the external database 201 to construct each complex system or mutually complex system. The data collection module 151 receives data for constructing the complex system from the user or collects food product data, human physiology data, environmental data, and health-related data in a data collecting manner from the database in which data related to the user's health is stored.

For example, the data collection module 151 may collect user's data, user personal information which are collected from a wearable device and a smart device, which are worn by the user, and the external database 201, the user's health-related data, food product data, human physiology data, and environmental data such as the data stored in a public database.

The food product data may include food ingredients such as nutritional ingredients of food, bioactive ingredients, functional ingredients, allergens, food additives, pesticide residues, pesticides, toxic substances, and pollutants, and data on food characteristics such as calorie density, portion content, glycemic index, color, taste, flavor, and texture of food.

In addition, the human physiology data may include data such as diseases, health, metabolites, proteomic bodies, transcriptomes, epigenes, genomes, intestinal microbes, microbiome, molecular physiological paths, and the like. The environmental data may include data related to climate, fine dust, smoking, hazardous substances, pollutants, and the like.

The user's health-related data means data related to personal health as a predictive variable for deriving the personalized diet. As a method for acquiring health-related data, any one or more of a method for acquiring the user's health-related data through a questionnaire, a method for measuring the user's body, a method for acquiring the health-related data using an analysis of a user's human body origin, and a method for using the user's health-related data collected by using the user's wearable device, the web, and the device, the web, and the app.

For example, when the health-related data is acquired through a questionnaire, the user may input, into the user's terminal, the user's personal information such as age, gender, height, weight, race, religion, residence, occupation, and food allergy items of the user, the user's personal health-related information such as memory, relaxation, sleep, cognitive function, fatigue, stomach, liver, bowel, body fat, absorption (calcium), male reproduction, female reproduction, kidney, urinary tract, hypersensitivity immunity, immunity, antioxidant, eyes, oral cavity, skin, triglyceride, cholesterol, blood pressure, blood circulation, blood sugar, hormones, joints, bones, muscles, children's height growth, sperm motility, and uric acid level, and information on the health goal. Therefore, the user may acquire the health-related data.

In addition, in a case of acquiring the health-related data through the user's body measurement and human-derived analysis, the health checkup results and hospital treatment records may be collected through the app, web, chatting consultation, video consultation, and phone consultation, or the health-related data may be acquired by using a method for collecting data from a database in which the user's health checkup results and hospital treatment records are stored.

Here, the health checkup results and hospital treatment records may include the checkup date, height, weight, waist circumference, body mass index, vision, hearing, blood pressure, urine protein, hemoglobin, pre-meal blood sugar, total cholesterol, HDL cholesterol, triglyceride, LDL cholesterol, serum creatinine, glomerular filtration rate, AST, ALT gamma GPT, osteoporosis, and the like.

In addition, the user's genetic data may be further included as one of the health-related data. The genetic data may mean a genetic test result, a risk genetic test result for a specific disease, or the like. The genetic data may be acquired by using a method for collecting data generated by using a gene analysis institution or a gene analysis kit through an app, web, chatting consultation, video consultation, and phone consultation, or inputting the data by using the user terminal 200 by the user, or a method for collecting the data from a database in which the user's genetic data is stored, and a database of a user's genetic data collection and analysis company.

The genetic data may include information on vitamin C concentration, vitamin D concentration, coenzyme Q10 concentration, magnesium concentration, zinc concentration, iron storage concentration, potassium concentration, calcium concentration, arginine concentration, fatty acid concentration, vitamin A, vitamin B6, vitamin B12, vitamin E, vitamin K, tyrosine, betaine, selenium, lutein, zeaxanthin, melasma, freckles, pigmentation, acne occurrence, skin aging, skin softening, appetite, satiety, sweetness sensitivity, bitter taste sensitivity, salty taste sensitivity, alcohol metabolism, alcohol dependence, alcohol hot flush, wine preference, caffeine metabolism, caffeine dependence, insomnia, obesity, triglyceride concentration, body fat percentage, body mass index, cholesterol, uric acid level, susceptibility to degenerative arthritis, blood sugar, blood pressure, bone mass, abdominal obesity, weight recovery possibility after weight loss, or the like.

In addition, the user's intestinal microbiome data, urine metabolite analysis data, hair mineral, and hair heavy metal analysis data included in the health-related data may be respectively collected by analyzed and measured results through the app, web, chatting consultation, video consultation, and phone consultation. In addition, the user's intestinal microbial flora data, urine metabolite analysis data, and hair mineral and hair heavy metal analysis data included in the health-related data may be acquired by using a method for inputting the data into the user terminal 200 by the user, or a method for collecting the data from a database in which the analyzed and measured data is stored.

In addition, for the method for collecting the health-related data through wearable devices, web, and apparatus, a method for collecting data such as weight, the number of steps, activity amount, exercise amount, heart rate, blood pressure, sleep, BMI, defecation status, and menstrual cycle, or acquiring the data from a database in which related data is stored may be used.

In order to construct the complexity system between data, the complexity system construction unit 150 may include a data classification module 152 and a complexity system construction module 153. The data classification module 152 classifies the food product data, human physiology data, and environmental data which are described above. The data classification module 152 may perform a function of classifying, standardizing, or standardizing the collected data, or performing quality management of the data.

In addition, the data classification module 152 may perform association between data. The data association may mean a function of associating data sets related to food, and food and nutritional ingredients, associating data sets related to food, and food and health function validity information, or associating data sets related to food and menu (diet).

The data classification module 152 may formulate and standardize data for each food product data, human physiology data, and environmental data. In addition, the data classification module 152 may perform a function of classifying data with high correlation or classifying data with low correlation among food product data, human physiology data, and environmental data. Here, the correlation means a case where one data affects a change of a value of another data by academic material, research material, or expert opinion.

The complex system construction module 153 constructs correlation between food product data, human physiology data, and environmental data classified by using the data classification module 152, and big data related to correlation thereof. At this time, rather than simply constructing the complex system by deriving the correlation between all food product data, human physiology data, and environmental data; using the user's health data are used. Construct the complex system is constructed by using food product data, human physiology data, and environmental data that are related to the user's health data by using the user' health-related data.

For example, when constructing the complex system between food product data and human physiology data, the complex system construction module 153 may construct the complex system using the following correlation between data.

The complex system construction module 153 may construct the complex system between food product data and human physiology data using food complex system correlation of nutritional ingredients and functional ingredients that affect interest health goals (human physiology complex such as memory, relaxation, sleep, cognitive function, fatigue, stomach, liver, intestine, body fat, absorption (calcium), male reproduction, female reproduction, kidney and urinary tract, hypersensitivity immunity, immunity, antioxidant, eyes, oral cavity (teeth), skin, triglyceride, cholesterol, blood pressure, blood circulation, blood sugar, hormone (menopausal health), joint/bone, muscle, height growth of children, sperm motility, and uric acid level) selected according to personal information, foods that contain a lot of them, and the food complex system of a specific menu.

The complex system construction module 153 may construct the complex system between food product data and human physiology data by using the food complex system correlation between nutritional ingredients and functional ingredients that affect the mechanism biomarkers and symptoms of diseases with a high family history, foods containing a lot of them, and food complex system correlation of a specific menu.

The complex system construction module 153 may construct the complex system between food product data and human physiology data by using the food complex system correlation between nutritional ingredients and functional ingredients that affect levels of height, weight, waist circumference, body mass index, visual acuity, hearing, blood pressure (systolic/diastolic), urine protein, hemoglobin, preprandial blood sugar, total cholesterol, HDL cholesterol, triglyceride, LDL cholesterol, serum creatinine, glomerular filtration rate, AST, ALT, gamma GPT, osteoporosis, and the like as results of health checkup, or affect the human physiological complex system of a specific disease as results of health checkup, foods containing a lot of them, and food complex system correlation of a specific menu.

Complex system construction module 153 may construct the complex system between food product data and human physiology data by using correlation between lifestyle habits such as eating habits (food material purchase data, delivery food order data, dining out data, preference/non-preference preference, contents, time of day, before and after meals (food eaten and leftover food), and food photography) fasting time, drinking, and smoking habits, and the human physiological complex system.

The complex system construction module 153 may construct a complex system between food product data and human physiology data by using correlation between postprandial blood glucose, blood lipid response, and biomarker.

The complex system construction module 153 may construct the complex system between food product data and human physiology data by using correlation between nutritional ingredients and functional ingredients that are required by individuals based on a personal health record (PHR) such as weight, the number of steps, activity amount, exercise amount, heart rate, blood pressure, sleep, BMI, defecation status, and menstrual cycle, for a certain period (1 week, 1 month, 1 year, or the like), foods containing a lot of them, and a food complex system of a specific menu.

The complex system construction module 153 may construct a complex system between food product data and human physiology data by using correlation between genetic information (human physiology complex system), nutritional ingredients and functional ingredients that affect the genetic information, foods containing a lot of them, and a food complex system of a specific menu.

The complex system construction module 153 may construct the complex system between food product data and human physiology data by using correlation between the intestinal microbial flora, postprandial blood glucose response, and postprandial blood lipid response.

The complex system construction module 153 may construct the complex system between food product data and human physiology data by using correlation between urine metabolites, nutritional ingredients and functional ingredients, foods containing a lot of them, and specific menu intake food complex system.

The complex system construction module 153 may construct the complex system between food product data and human physiology data by using correlation between hair minerals & heavy metals, nutritional ingredients and functional ingredients, foods containing a lot of them, and specific menu intake food complex system.

In addition, when constructing the complex system by using human physiology data and environmental data, the complex system construction module 153 may construct the complex system by using the following correlation between data.

The complex system construction module 153 may construct the complex system between human physiology data and environmental data by using correlation of an environmental complex system that affects interest health goals (human physiology complex such as memory, relaxation, sleep, cognitive function, fatigue, stomach, liver, intestine, body fat, absorption (calcium), male reproduction, female reproduction, kidney and urinary tract, hypersensitivity immunity, immunity, antioxidant, eyes, oral cavity (teeth), skin, triglyceride, cholesterol, blood pressure, blood circulation, blood sugar, hormone (menopausal health), joint/bone, muscle, height growth of children, sperm motility, and uric acid level) selected according to personal information.

The complex system construction module 153 may construct the complex system between human physiology data and environmental data by using correlation of an environmental complex system that affects the mechanism biomarkers and symptoms of diseases with a high family history.

The complex system construction module 153 may construct the complex system between human physiology data and environmental data by using correlation of an environmental complex system that affects levels of height, weight, waist circumference, body mass index, visual acuity, hearing, blood pressure (systolic/diastolic), urine protein, hemoglobin, preprandial blood sugar, total cholesterol, HDL cholesterol, triglyceride, LDL cholesterol, serum creatinine, glomerular filtration rate, AST, ALT, gamma GPT, osteoporosis, and the like as results of health checkup, or affects the human physiological complex system of a specific disease as results of health checkup.

The complex system construction module 153 may construct the complex system between human physiology data and environmental data by using correlation of genetic information (human physiology complex system) and the environmental complex system that may epigenetically affect thereon.

The complex system construction module 153 may construct the complex system between human physiology data and environmental data by using correlation between urine metabolites and environment-exposed environmental complex system.

The complex system construction module 153 may construct the complex system between human physiology data and environmental data by using correlation between hair minerals & heavy metals and environment-exposed environmental complex system.

The complex system construction module 153 may construct the complex system between human physiology data and environmental data by using a recommended intake amount and an upper limit intake amount of nutritional ingredients and functional ingredients according to age and gender, and drug interactions thereof.

The complex system construction module 153 may construct the complex system by using the same data as the user's health-related data or data having a similarity or relevance to the user's health-related data more than a preset reference value in addition to constructing the complex system among all the collected data to construct the complex system.

The complex system has a non-linear relationship between a wide variety of data. Therefore, simply constructing the complex system between all data dramatically increases the cost and time for constructing the complex system. The complex system construction module 153 constructs the complex system by selectively using the same data as the user's health-related data or data having a similarity or relevance to the user's health-related data equal to or greater than a preset reference value. Accordingly, it is possible to reduce the cost and time required to construct the complex system, and to construct a database more suitable for the user.

The complex system construction module 153 may set each of a plurality of variables included in food product data, human physiology data, and environmental data as one node. In addition, the complex system may be constructed by learning the correlation or association between respective nodes. In addition, the complexity system construction module 153 may set that the nodes to have the association only when the correlation or association between nodes is equal to or greater than a preset threshold value.

In addition, the complex system construction module 153 may generate a plurality of data sets by classifying variables having the same meaning or high relevance among a plurality of variables included in food product data, human physiology data, and environmental data, and set each data set to a node. In addition, a complex system may be constructed by learning the correlation or association between respective nodes. In addition, the complexity system construction module 153 may set the correlation or association between nodes as having a relevance between the nodes only when the correlation or association between the nodes is equal to or greater than a preset threshold value.

The complex system construction module 153 may perform learning by using any one or more of supervised learning, unsupervised learning, semi-supervised learning, and reinforcement learning to construct a complex system. In addition, the complex system construction module 153 may use any one or more of neural network structures such as an artificial neural network (ANN), a deep neural network (DNN), a convolution neural network (CNN), and a recurrent neural network (RNN) to construct a complex system.

The personalized dietary derivation unit 160 may predict health maintenance, disease prevention nutritional ingredients, and physiologically active substances (functional ingredients) tailored to individual health-related characteristics, foods containing a lot of them, and specific menu by using the database of food product data, human physiology data, and environmental data constructed in the complexity system construction unit 150 and the user's health-related data. In this case, the personalized dietary derivation unit 160 may derive a user-personalized diet by performing machine learning.

The personalized dietary derivation unit 160 not only recommends health maintenance, disease prevention nutritional ingredients, and physiologically active substances (functional ingredients) tailored to individual health-related characteristics of the user, and foods containing a lot of them, but also a method for preparing or consuming the food product to maximize personalized nutritional ingredients.

In addition, the personalized dietary derivation unit 160 may assign importance to health and disease prevention substances in predicted nutrients, physiologically active substances (functional raw materials), foods, and menu, and also predict the importance to food materials including them based thereon.

The personalized dietary derivation unit 160 may construct a genome-wide activity profile by simulating operations of health maintenance and disease prevention substances having clinical evidence in human interactome.

The personalized dietary derivation unit 160 may use a list of food ingredients identified through model design and systematic review as the learning data, by using an approaching method which is supervised to predict health function and disease prevention-related food ingredients by using a learned interactive activity profile. In addition, the personalized dietary derivation unit 160 may apply and compare various machine learning models such as k-nearest neighbors (kNN), decision tree (DT), support vector machine (SVM), and moving morphable component (MMC).

The personalized dietary derivation unit 160 may predict substances that will show health and disease prevention effects from a database of nutrients and physiologically active substances (functional raw materials) in food by using the designed model.

For example, the personalized dietary derivation unit 160 may perform nutritional evaluation of a diet to be consumed by the user by using food, ingredients, and nutritional ingredients included in the food. The personalized dietary derivation unit 160 may provide the user-personalized diet by using user's personal information such as diseases, disease occurrence potential, allergens, genetic information data and human body, genes, allergies, preferences/tastes, intestinal microbes, and dietary behaviors according to nutritional ingredients by using the derived nutritional evaluation.

In addition, the personalized dietary derivation unit 160 may classify diseases into 11 types, such as depression, hypertension, diabetes, obesity, hyperlipidemia, gastric cancer, liver cancer, lung cancer, colorectal cancer, breast cancer, and cervical cancer. The personalized dietary derivation unit 160 may provide a personalized diet by using disease information of the 11 types.

The personalized dietary derivation unit 160 may derive a suitability rate indicating how suitable food is for each disease by using research results of nutritional ingredients that affects the disease-specific pathogenesis, occurrence factors, and diet.

The personalized dietary derivation unit 160 may derive an applied nutrient level according to disease by considering a daily recommended amount for Koreans as a reference value by selecting 16 applied nutrients such as carbohydrates, proteins, fats (saturated fats and unsaturated fatty acids), tryptophan, cholesterol, vitamin A, vitamin B, vitamin C, vitamin E, selenium (Se), magnesium (Mg), calcium (Ca), sodium (Na), and dietary fiber.

In the case of depression, an appropriate ratios of carbohydrates, proteins, fats, tryptophan, saturated fatty acids, cholesterol, vitamin B, selenium, magnesium, and calcium may be derived. In addition, in the case of high blood pressure, an appropriate ratio of carbohydrates, proteins, fats, sodium, dietary fiber, saturated fatty acid, potassium, magnesium, and calcium may be derived.

In addition, in the case of diabetes, an appropriate ratio of carbohydrates, proteins, fats, dietary fiber, and saturated fatty acid may be derived, and in the case of obesity, an appropriate ratio of carbohydrates, proteins, fats, and dietary fiber may be derived. In addition, in the case of hyperlipidemia, an appropriate ratio of carbohydrates, proteins, fats, saturated fatty acid, unsaturated fatty acid, fiber, vitamin C, vitamin E, and calcium may be derived.

In addition, in the case of stomach cancer, an appropriate ratio of carbohydrates, proteins, fats, sodium, unsaturated fatty acid, vitamin C, and vitamin E may be derived, and in the case of lung cancer, an appropriate ratio of carbohydrates, proteins, fats, vitamin A, vitamin C, and vitamin E may be derived. In addition, in the case of liver cancer, an appropriate ratio of carbohydrates, proteins, fats, vitamin A, vitamin C, and vitamin E may be derived.

In addition, in the case of cervical cancer, an appropriate ratio of carbohydrates, proteins, fats, vitamin A, vitamin C, and vitamin E may be derived, in the case of breast cancer, an appropriate ratio of carbohydrates, proteins, fats, vitamin A, and vitamin C may be derived, and in the case of colorectal cancer, an appropriate ratio of carbohydrates, proteins, fats, fiber, lactose, and vitamin C may be derived.

The personalized dietary derivation unit 160 may derive an appropriate ratio of nutrients by classifying intake nutrient appropriate value, for class interval, by adding or subtracting an applied value for each intake nutrient according to the disease based on the daily intake nutrient and nutritional recommendation for Koreans in order to derive the appropriate ratio of the nutrient according to the disease.

For example, tryptophan is a nutrient suitable for depression, and a recommended ratio for adults may be set at 300 to 400 mg per day. In the case of cholesterol, a recommended ratio for adults may be set at 200 to 400 mg per day, and saturated fatty acid may be set at 4.5 to 7% of an average daily recommended calorie for adults (2600 kcal for men, 2100 kcal for women).

In addition, vitamins B3, B5, and B6 may be set at 100 to 500 mg, vitamin B9 at 400 to 800 μg, and vitamin B12 at 100 to 500 μg. Selenium may be set at 55 to 100 μg, magnesium at 280 to 340 μg, and calcium at 700 to 1000 mg.

In addition, if the user is a depressed patient, a value 10% reduced from the adult standard recommended ratio of saturated fatty acid and cholesterol, which increase the risk of developing depression, may be suggested. In addition, tryptophan, vitamin B, selenium, magnesium, and calcium, which have a positive effect on depression, may suggest a value 10% increased in the recommended ratio for adults.

For example, the personalized dietary derivation unit 160 may perform nutritional evaluation of a menu to be consumed by the user by using food, material, and nutritional ingredients included in the food. The personalized dietary derivation unit 160 may provide the user-personalized diet by using user' personal information such as diseases, disease occurrence potential, allergens, genetic information data and human body, genes, allergies, preferences/tastes, intestinal microbes, and dietary behaviors according to nutritional ingredients by using the derived nutritional evaluation.

The personalized dietary derivation unit 160 may perform performance verification of a predictive model. To this end, the personalized dietary derivation unit 160 may verify the derivation result with a k-fold cross-validation evaluation method or may evaluate derivation performance by using an area under the ROC Curve (AUC) and F1 score. The personalized dietary derivation unit 160 may demonstrate the sensitivity and specificity of the result of derivation of the personalized diet in a platform through a cross validation and a receiver operation characteristic curve, or the like.

The personalized dietary derivation unit 160 may perform learning to derive the personalized diet by using any one or more of supervised learning, unsupervised learning, semi-supervised learning, and reinforcement learning, which are classified according to the supervised form or amount of information during learning in the classification of machine learning.

For example, the personalized dietary derivation unit 160 may perform supervised learning on a data set having correlation or relevance which is equal to or greater than a first threshold value among data included in the database constructed by the complexity system construction unit 150. In addition, the personalized dietary derivation unit 160 may perform semi-supervised learning with respect to a data set having correlation or relevance which is equal to or greater than a second threshold value among data included in the database constructed by the complexity system construction unit 150. In addition, the personalized dietary derivation unit 160 may perform unsupervised learning or reinforcement learning with respect to a data set having correlation or relevance which is less than a third threshold value among data included in the database constructed by the complexity system construction unit 150 to increase the learning efficiency of the personalized dietary derivation unit 160.

As described above, the personalized dietary suggestion platform providing system 100 may perform machine learning to construct the complex system or derive the personalized diet by using the user's health-related data, food product data, human physiology data, and environmental data.

The personalized dietary suggestion platform providing system 100 may use K-Nearest Neighbors, Linear Regression, Logistic Regression, Support Vector Machines (SVM), a Decision Tree, a Random Forest, and a Neural Networks to perform supervised learning.

In addition, the personalized dietary suggestion platform providing system 100 may use any one or more of clustering, unsupervised/supervised hybrid clustering, K-Means, Hierarchical Cluster Analysis (HCA), Expectation Maximixation, Visualization, dimensionality reduction, Principal Component Analysis (PCA), Kernel PCA, Locally-Linear Embedding (LLE), t-distributed Stochastic Neighbor Embedding (t-SNE), Association rule learning, Apriori, and Eclat to perform unsupervised learning.

The personalized dietary suggestion platform providing system 100 may use any one or more of neural network structures such as an Artificial Neural Network (ANN), a Deep Neural Network (DNN), a Convolution Neural Network (CNN), and a Recurrent Neural Network (RNN), as a method for performing machine learning.

The ANN may include an input layer, a hidden layer, and an output layer. In the input layer, data for constructing the database of the complexity system, data derived by using the complexity system construction unit 150, or the complexity system data constructed by using the complexity system construction unit 150 are input.

The hidden layer sets a weight to the data input from the input layer. In addition, a result value is derived by changing the binding strength and weight of the synapse through repeated learning. In the output layer, a complex system construction result or a personalized diet according to the learning result may be output. DNN may improve the learning result by increasing the number of hidden layers more than that of ANN.

The CNN may be configured by using a convolution layer, a pooling layer, a fully connected layer, and an output layer. The convolution layer derives the characteristics of the input data using the ReLU function. That is, it is possible to derive the characteristics of the user's health-related data, food product data, human physiology data, and environmental data for constructing the complex system and deriving the personalized diet. The pooling layer may reduce the size of data by canceling noise of data that has passed through the convolution layer and performing normalization.

The product recommendation unit 170 recommends personalized diet and products using the results derived from the personalized dietary derivation unit 160. The Diet and products recommended by the product recommendation unit 170 may include menu, agricultural and livestock products, processed food, cooked food, meal kits, cooking method provision, agricultural and livestock products, processed foods, cooked foods, meal kits, recipes, dining out, health functional foods/nutrients, smart farms, cosmetics, drugs, herbal medicines, and the like.

The product recommendation unit 170 may provide the user with a personalized dietary product purchase list including any one or more of food materials, food, nutritional supplements, and medicines. In this case, the order of products displayed on the list may be set according to a preset criterion or weight.

The product recommendation unit 170 may suggest the user's food to be wary of, fasting time, activity amount, heart rate, and the like. In addition, the product recommendation unit 170 may provide a service such that dietary recommendation and purchase may be directly connected in connection with an insurance company and a seller of the recommended diet and product. That is, the product recommendation unit 170 may provide the user with products and services such as food, cosmetics, health care service, and insurance, based on the derived personalized diet result.

In addition, the product recommendation unit 170 may transmit the personalized dietary list to a retailer mainly used by the user or a convenience store located in the customer's movement. Accordingly, the retailer and the convenience store may prepare products included in the user's personalized dietary list and deliver them to the user, or the user may pick up the products.

The personalized dietary suggestion program may perform feedback through scientific and clinical review by experts on the results of the complex system construction unit 150 and the personalized dietary derivation unit 160. Accordingly, demonstration and feedback on whether scientific validation for personalized dietary suggestion programs and the suggested diet are capable of being realized and effective are possible.

For example, the personalized dietary suggestion program may transmit correlation data between food product data, human physiology data, and environmental data derived from the complex system construction unit 150 and the personalized dietary derivation unit 160, and the personalized dietary derivation results to the expert. In addition, by receiving expert opinions as answers, feedback on whether there are erroneous calculated results is performed, so that it is possible to correct correlation data between food product data, human physiology data, and environmental data, and personalized dietary derivation results.

In addition, the personalized dietary suggestion program may secure equal to or greater than 1,000 participants per target group using the app or web for collecting personal health data and provide feedback on the responses to the personalized diet designed by the expert group and the personalized dietary suggestion program. Accordingly, the personalized dietary suggestion program may learn to derive results similar to the personalized diet derived by an expert.

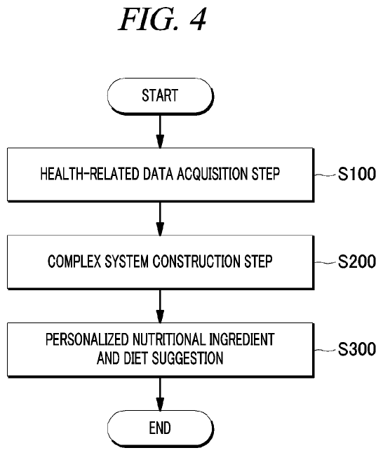

Hereinafter, a method for providing a personalized dietary suggestion platform according to an embodiment of the present disclosure will be described with reference to FIG. 4.

As shown in FIG. 4, it may include a health-related data acquisition step (S100), a complex system construction step (S200), and a personalized nutritional ingredient and diet suggestion step (S300).

In the health-related data acquisition step (S100), user's health-related data is collected such as the user's data and user personal information collected from the wearable device, smart device, and health-related data collection, which are worn by the user, analysis company 200, and external database 201, and data data stored in the public database.

As the method for acquiring the health-related data as described above with reference to FIG. 3, any one method or more of a method for acquiring user' health-related data through a questionnaire, a method for measuring the user's body, a method for acquiring user's health-related data using a user's human body derivative analysis, a method for using user's health-related data through a user's wearable device, web and, apparatus, and a method for using data from a health-related data collection and analysis company may be used.

In the complex system construction step (S200), the complex system may be constructed by using interactions and correlation between user's health-related data, food product data, human physiology data, and environmental data.

In the complex system construction step (S200), any one or more of classification, formulation, and standardization may be performed with respect to food product data, human physiology data, and environmental data. In addition, the complex system may be constructed by deriving correlation between processed food product data, human physiology data, and environmental data.

As described above, in the complex system construction step (S200), each of a plurality of variables included in the food product data, the human physiology data, and the environment data may be set as one node. In addition, the complex system may be constructed by learning the correlation or association between respective nodes. In addition, in the step of constructing a complex system (S200), the nodes may be set as having correlation therebetween only when the correlation or association between the nodes is equal to or greater than a preset threshold value.

In addition, in the complex system construction step (S200), a plurality of data sets may be generated by classifying variables having the same meaning or high relevance among a plurality of variables included in food product data, human physiology data, and environmental data, and assign each data set may be set to a node. In addition, the complex system may be constructed by learning the correlation or association between respective nodes. In addition, the complexity system construction module 153 may set the correlation or association between nodes as having a relevance between the nodes only when the correlation or association between the nodes is equal to or greater than a preset threshold value.

In the personalized nutritional ingredient and dietary suggestion step (S300), health maintenance, disease prevention nutritional ingredients, and physiologically active substances (functional ingredients) tailored to individual health-related characteristics, foods containing a lot of them, and specific menu may be derived by using the complexity system related to food product data, human physiology data, and environmental data and the user's health-related data.

In addition, in the personalized nutritional ingredient and dietary suggestion step (S300), a list of recommended purchases for food, medicine, cosmetics, healthcare products, healthcare services, insurance, and the like may be provided to the user, or a function of relaying between a seller (distributor and insurance companies) and the user may be performed.

For example, in the personalized nutritional ingredient and dietary suggestion step (S300), the seller 300 may be displayed preferentially, to the user, based on preset criteria or according to the weight to suggest a purchase of food, medicine, and cosmetics, by using the result of the personalized dietary suggestion, or the product of the seller 300 registered in the personalized dietary suggestion platform providing system 100 may be provided to the user.

In the personalized nutritional ingredients and dietary suggestion step (S300), the personalized nutritional ingredients or the food product including the personalized nutritional ingredients may be derived by using any one or more of k-nearest neighbors (kNN), decision tree (DT), support vector machine (SVM), and moving morphable component (MMC).

In the personalized nutritional ingredient and dietary suggestion step (S300), importance may be assigned to health and disease prevention substances in derived nutrients, physiologically active substances (functional raw materials), foods, and menu, and also the importance to food materials including them may be predicted based thereon.

In addition, in the personalized nutritional ingredient and diet suggestion step (S300), a method for preparing or consuming the food product to maximize personalized nutritional ingredients may be recommended with respect to the food product including the personalized nutritional ingredients. In addition, in the personalized nutritional ingredient and diet suggestion step (S300), the personalized dietary product purchase list including any one or more of food materials, food, nutritional supplements, and medicines may be provided to the user.

In addition, in the personalized nutritional ingredients and dietary suggestion step (S300), in order to increase the reliability of the user, the sensitivity and specificity of the personalized dietary derivation result may be derived by using any one or more of cross validation, receiver operation characteristic curve, and F1 score.

In the personalized nutritional ingredient and diet suggestion step (S300), feedback through scientific and clinical review by experts on the personalized dietary derivation result may be performed. Accordingly, demonstration and feedback on whether scientific validation for personalized dietary suggestion programs and the suggested diet are capable of being realized and effective are possible.

For example, in the personalized nutritional ingredient and diet suggestion step (S300), correlation data between food product data, human physiology data, and environmental data derived from the complex system construction unit 150 and the personalized dietary derivation unit 160, and the personalized dietary derivation results may be transmitted to the expert. In addition, by receiving expert opinions as answers, feedback on whether there are erroneous calculated results is performed, so that it is possible to correct correlation data between food product data, human physiology data, and environmental data, and personalized dietary derivation results.

In addition, in the personalized nutritional ingredient and dietary suggestion step (S300), equal to or greater than 1,000 participants per target group using the app or web for collecting personal health data may be secured, and feedback may be provided on the responses to the personalized diet designed by the expert group and the personalized dietary suggestion program. Accordingly, the personalized dietary suggestion program may learn to derive results similar to the personalized diet derived by an expert.

An embodiment of the present disclosure may be implemented in a form of a recording medium including instructions executable by a computer, such as a program module executed by a computer. Computer-readable media may be any available media that may be accessed by a computer and includes all volatile and nonvolatile media, removable and non-removable media. In addition, computer-readable media may include computer storage media. Computer storage media includes all volatile and nonvolatile, removable and non-removable media implemented in any method or technology for storage of information such as computer readable instructions, data structures, program modules or other data.

Although the method and system of the present disclosure have been described with reference to specific embodiments, some or all of their components or operations may be implemented by using a computer system having a general purpose hardware architecture.

The description of the present disclosure described above is for illustration, and those of ordinary skill in the art to which the present disclosure pertains may understand that it may be easily modified into other specific forms without changing the technical spirit or essential features of the present disclosure. Therefore, it should be understood that the embodiments described above are illustrative in all respects and not restrictive. For example, each component described as a single type may be implemented in a dispersed form, and likewise components described as distributed may also be implemented in a combined form.

The scope of the present disclosure is indicated by the following claims rather than the above detailed description, and all changes or modifications derived from the meaning and scope of the claims and their equivalent concepts should be interpreted as being included in the scope of the present disclosure.

What is claimed is:

1. A method for providing a personalized dietary suggestion platform performed by a processor, comprising:
a step of acquiring health-related data related to the user's health;
a step of constructing a database by using big data including food product data, human physiology data, and environmental data, and the health-related data; and
a step of suggesting a personalized nutritional ingredient including any one or more of a health maintenance ingredient, a disease nutritional ingredient, and a physiologically active ingredient tailored to user's health-related characteristics, or a food product including the personalized nutritional ingredient by executing a personalized dietary derivation model learned by using the database,
wherein the food product data includes data related to a nutritional ingredient of a food product and characteristics of the food product, the human physiology data includes data related to body, disease, and genetic information, and the environmental data includes data related to a climate environment and an environmentally hazardous substance.

2. The method for providing a personalized dietary suggestion platform of claim 1,
wherein the step of acquiring the health-related data includes,
a step of acquiring data related to a user's health from any one or more of a user's smart device a wearable device, and an external database, and
wherein the external database means a server or a database in which data acquired from a user's wearable device, a smart device, and a health-related data collection and analysis company, user's health checkup history data, or user's health-related data is stored.

3. The method for providing a personalized dietary suggestion platform of claim 1,
wherein the step of constructing the database includes:
a step of performing any one or more of classification, formulation, and standardization related to the food product data, the human physiology data, and the environmental data, and
a step of constructing a complex system by deriving a correlation between the food product data, the human physiology data, and the environmental data.

4. The method for providing a personalized dietary suggestion platform of claim 1,
wherein the step of suggesting a personalized nutritional ingredient or a food product including the personalized nutritional ingredient includes:
a step of recommending a method for preparing or consuming the food product to maximize the personalized nutritional ingredient with respect to the food product including the personalized nutritional ingredient.

5. The method for providing a personalized dietary suggestion platform of claim 1,
wherein the step of suggesting a personalized nutritional ingredient or a food product including the personalized nutritional ingredient further includes:
a step of providing a personalized dietary product purchase list including any one or more of a food material, food, a nutritional supplement, and a medicine to a user.

6. The method for providing a personalized dietary suggestion platform of claim 1,
wherein the step of suggesting a personalized nutritional ingredient or a food product including the personalized nutritional ingredient further includes:
a step of deriving the personalized nutritional ingredient or the food product including the personalized nutritional ingredient by using any one or more of k-nearest neighbors (kNN), decision tree (DT), support vector machine (SVM), and moving morphable component (MMC).

7. The method for providing a personalized dietary suggestion platform of claim 1,
wherein the step of suggesting a personalized nutritional ingredient or a food product including the personalized nutritional ingredient further includes:
a step of deriving a sensitivity and a specificity of a personalized dietary derivation result by using any one or more of a cross validation, a receiver operation characteristic curve, and an F1 score.

8. A system for providing a personalized dietary suggestion platform, comprising:
a memory in which a personalized dietary suggestion program is stored; and
a processor that executes the personalized dietary suggestion program stored in the memory,
wherein the processor executes the personalized dietary suggestion program,
acquires health-related data related to user's health,
constructs a database using big data including food product data, human physiology data, and environmental data, and health-related data, and
suggests a personalized nutritional ingredient including any one or more of a health maintenance ingredient, a disease nutritional ingredient, and a physiologically active ingredient tailored to a health-related characteristic of the user or a food product including the personalized nutritional ingredient by executing a personalized dietary derivation model learned by using the database, and
wherein the food product data includes data related to a nutritional ingredient and a characteristic of the food product, the human physiology data includes data related to a body, a disease, and genetic information, and the environmental data includes data related to a climatic environment and an environmentally hazardous substance.

9. The system for providing a personalized dietary suggestion platform of claim 8,
wherein the processor executes the personalized dietary suggestion program,
to acquire data related to a user's health from any one or more of a user's smart device a wearable device, and an external database, and
wherein the external database means a server or a database in which data acquired from a user's wearable device, a smart device, and a health-related data collection and analysis company, user's health checkup history data, or user's health-related data is stored.

10. The system for providing a personalized dietary suggestion platform of claim 8,
wherein the processor executes the personalized dietary suggestion program,
to perform any one or more of classification, formulation, and standardization related to the food product data, the human physiology data, and the environmental data, and construct a complex system by deriving a correlation between the food product data, the human physiology data, and the environmental data.

11. The system for providing a personalized dietary suggestion platform of claim 8,
wherein the processor executes the personalized dietary suggestion program,
to recommend a method for preparing or consuming the food product to maximize the personalized nutritional ingredient with respect to the food product including the personalized nutritional ingredient.

12. The system for providing a personalized dietary suggestion platform of claim 8,
wherein the processor executes the personalized dietary suggestion program,
to provide a personalized dietary product purchase list including any one or more of a food material, food, a nutritional supplement, and a medicine to a user.

13. The system for providing a personalized dietary suggestion platform of claim 8,
wherein the processor executes the personalized dietary suggestion program,
to derive the personalized nutritional ingredient or the food product including the personalized nutritional ingredient by using any one or more of k-nearest neighbors (kNN), decision tree (DT), support vector machine (SVM), and moving morphable component (MMC).

14. The system for providing a personalized dietary suggestion platform of claim 8,
wherein the processor executes the personalized dietary suggestion program,
to derive a sensitivity and a specificity of a personalized dietary derivation result by using any one or more of a cross validation, a receiver operation characteristic curve, and an F1 score.
